(19) United States
(12) Patent Application Publication
(10) Pub. No.: US 2024/0024689 A1
(43) Pub. Date: Jan. 25, 2024

(54) STACK DESIGN IMPLANT DEVICE

(71) Applicant: Nyxoah SA, Mont Saint Guibert (BE)

(72) Inventors: Koen Erik VAN DEN HEUVEL, Hove (BE); Jan VERMEIREN, Boechout (BE); Wim BERVOETS, Wilrijk (BE); Martin Joseph SVEHLA, Botany (AU); Milind Chandrakant RAJE, Glenwood (AU); Tom MEEUSEN, Kessel-lo (BE)

(73) Assignee: Nyxoah SA, Mont Saint Guibert (BE)

(21) Appl. No.: 18/253,261

(22) PCT Filed: Nov. 18, 2020
(86) PCT No.: PCT/EP2020/082564

§ 371 Date: May 17, 2023

(51) Int. Cl. A61N 1/375; A61N 1/378; A61N 1/36; A61N 1/04

(52) U.S. Cl. CPC A61N 1/3754; A61N 1/3787; A61N 1/36125; A61N 1/0476

(57) ABSTRACT

A modular implant device configured for stimulation of at least one nerve or muscle in a body of a subject comprises: a housing; and at least one electrical lead, and/or at least one stimulation electrode; wherein the at least one electrical lead is partly disposed on the housing, and wherein the housing comprises modular elements, the modular elements being arranged in a stack. A system for electrical nerve stimulation comprises a network of at least two modular devices configured for implantation inside a body of a subject according to one of the preceding claims, wherein each modular device is electrically connected to at least one other modular device through electrical leads.

### TECHNICAL FIELD

The disclosed subject matter described hereafter refers to a device for electrical nerve stimulation. Furthermore, reference is made to a use of the device for electrical nerve stimulation to correct sleep disordered breathing.

Neural modulation, e.g. electrical stimulation of nerves, is known in the prior art as a reliable and effective type of medical treatment. It presents the opportunity to tackle many physiological conditions and disorders by interacting with the body's own natural neural processes. Neural modulation includes inhibition (e.g. blockage), stimulation, modification, regulation, or therapeutic alteration of activity, electrical or chemical, in the central, peripheral, or autonomic nervous system. By modulating the activity of the nervous system, several different goals may be achieved. For instance, motor neurons may be stimulated at appropriate times to cause muscle contractions. Further, sensory neurons can be blocked to relieve pain or stimulated to provide a signal to a subject or patient. In yet other examples, modulation of the autonomy nervous system may be used to adjust various involuntary physiological parameters, such as heart rate and blood pressure. Neural modulation may provide the opportunity to treat several diseases or physiological conditions. Various devices and techniques have been used in attempts to provide optimum stimulation of a tissue of interest.

One of the conditions to which neural modulation can be applied to is obstructive sleep apnea (OSA), a respiratory disorder characterized by recurrent episodes of partial or complete obstruction of the upper airway during sleep. One of the causes of OSA is the inability of the tongue muscles to resist negative inspiratory pressure in the pharynx due to the sleep-related loss in muscle tone. As the tongue is pulled backwards, it obstructs the upper airway, decreasing ventilation and lowering lung and blood oxygen levels. Stimulation of the hypoglossal nerve for, example, causes the tongue muscles, e.g. the genioglossus muscle, to contract, thereby maintaining an open, unobstructed airway, since the genioglossus muscle is responsible for the forward movement of the tongue as well as for the stiffening of the anterior pharyngeal wall.

Another condition to which neural modulation may be applied is the occurrence of migraine headaches. Pain sensation in the head is transmitted to the brain via the occipital nerve, specifically the greater occipital nerve, and the trigeminal nerve. When a subject experiences head pain, such as during a migraine headache, the inhibition of these nerves may serve to decrease or eliminate the sensation of pain.

Neural modulation may also be applied to hypertension. Blood pressure in the body is controlled via multiple feedback mechanisms. For example, baroreceptors in the carotid body in the carotid artery are sensitive to blood pressure changes within the carotid artery. The baroreceptors generate signals that are conducted to the brain via the glossopharyngeal nerve when blood pressure rises, signaling the brain to activate the body's regulation system to lower blood pressure, e.g. through changes to heart rate, and vasodilation/vasoconstriction. Conversely, parasympathetic nerve fibers on and around the renal arteries generate signals that are carried to the kidneys to initiate actions, such as salt retention and the release of angiotensin, which raise blood pressure. Modulating these nerves may provide the ability to exert some external control over blood pressure.

The aforementioned are just a few examples of conditions to which neuromodulation may be of benefit. However, embodiments of the disclosure described hereafter are not limited to treating only the above-described conditions.

Prior Art

According to conventional solutions, the application range of an implant device used for functional electrical stimulation (FES) is limited to only few areas, since it often lacks functional and structural flexibility. Furthermore, conventional devices using lead electrodes for stimulation are often susceptible to lead damages due to inevitable movement of parts of the device, thus leading to low durability of the device.

### SUMMARY OF THE DISCLOSED SUBJECT MATTER

One of the objectives of the present disclosure is to respond to the demands of different areas of application of FES and to provide an improved device and system for electrical nerve or muscle stimulation in a patient or recipient. In particular, an objective of this disclosure is to present a device that can be used for a wide range of applications in FES. The device and the system should allow for flexible arrangement while being easy to manufacture as well as highly durable.

According to one aspect of the disclosure, a modular stimulation device configured for implantation inside a body of a subject is provided, the device comprising: a housing, and at least one electrical lead and/or at least one stimulation electrode, wherein the at least one electrical lead is partly attached to the housing, and wherein the housing comprises modular elements, the modular elements being arranged in a stack. Such a modular design is beneficial, since it is applicable for different product variants of stimulation devices. Furthermore, a stack design has the advantage of simplifying otherwise intricate manufacturing processes. Also, since the modular elements are preferably standardized, the manufacturing process can be accelerated significantly. Preferably, the stack comprises at least two modular elements attached to each other along a vertical axis, wherein the individual modular elements may be attached to each other via gluing, brazing, welding, or by any other hermetic fixation system.

The at least two modular elements may thereby consist of one electrically active module and one passive module (i.e. without ceramics), or they may comprise of two electrically active modules. In case of two electrically active modules, the two modules may be electrically connected with each other internally (e.g. in a hermetic cavity generated by the modules). It is also possible to stack the modular elements in an interchangeable order, allowing for an even higher flexibility in application. The modular implant device as described herein is applicable in any appropriate FES context. For example, the modular implant device may be used for stimulation therapies in cases of head or neck pain, spinal cord injuries, stroke and upper limb recovery, drop foot and hypoglossal stimulation.

According to one embodiment disclosed herein, each modular element comprises a frame, wherein said frame may furthermore be four sided, each side having a width. However, the frame may be of any shape, e.g. round. As part of a preferred embodiment, the frame may at least partly made from a titanium alloy. Additionally, titanium is biocompatible and can therefore be used for implantation in a subject's body without the risk of being rejected or causing inflammations or allergies. Further, titanium alloys are highly durable, long lasting, light weight, without compromising strength. Providing frames for the modular elements drastically increases stability of the modular elements and thus of the housing and the modular implant device, respectively. In addition, the frames facilitate manufacturing of the modular implant device, since they provide a working surface for assembling the modular elements to a stack. Furthermore, they allow handling of the modular elements without having to operating any intricate components.

The modular device may, in particular, be configured in such a way that at least one modular element is a strain relief module, wherein the strain relief module comprises a cavity enclosed by the frame and wherein the frame is configured as a strain relief ring. Such a strain relief module has the advantage of increasing the durability of the modular device, since it drastically minimizes the risk of lead damages. To further increase durability and to hermetically seal the housing, the cavity enclosed in the frame of the strain relief module may be backfilled with an insulating material, wherein the insulating material may comprise silicone. Silicone is flexible and biocompatible and therefore highly suitable for any application in FES.

Further to the above, the at least one electrical lead may comprise an electrical contact tip on at least one of its ends. It may then be preferable that the electrical contact tip is disposed in the housing. Each electrical lead is configured to electrically communicate signals from another circuit or from a power supply. According to yet another beneficial improvement, the electrical lead passes through a lead exit disposed in at least one of the sides of the strain relief ring of the strain relief module. Preferably, the width of a side of the strain relief ring having a lead exit is bigger than the width of a side of the strain relief ring having no lead exit. This way, the strain relief module provides more space for the electrical lead to pass through, including a strain relief functionality. For example, the wider side allows for the electrical lead to form a double bend when passing through the lead exit of the strain relief ring. Such a double bend prevents the lead from losing contact to a electric contact or a feedthrough of its respective circuit board or from being pulled out of the housing when accidently experiencing a tug force, e.g. from movement of the device or the electrical lead inside the body. Furthermore, the above concept allows for maximum freedom of electrical contact tips and lead exit position and orientation, since the strain relief ring can easily adapt for any lead exit location or orientation. This provides the modular device with a high flexibility with regards to applications in FES.

It may also be intended that at least one of the electrical leads is a stimulation lead configured for electrical stimulation of a nerve of the subject, or a power lead configured for delivering power from a power supply, or a connection lead configured for electrically connecting the modular device to another electrical device. According to a preferred embodiment, each stimulation lead comprises a tip electrode at one of its ends that is disposed on an outer surface of the housing of the modular device, electrically connected with the feedthrough, but that is positioned in a desired location with respect to the tissue to be stimulated. The stimulation of tissue using stimulation leads (e.g. in a location further away from the housing) can also be referred to as lead stimulation.

According to another example of the modular device, the at least one stimulation electrode is disposed on an outer surface of a housing side of the modular device. It may furthermore be intended that the modular device comprises a plurality of stimulation electrodes, wherein the plurality of stimulation electrodes is preferably arranged in an array. Theoretically, the number of electrodes arranged on the surface of the housing is only limited by the size of the modular implant device. An array being very densely packed with stimulation electrodes is for example suitable for being used in scanning applications. In order to provide stimulation to a desired tissue, the modular device may also be equipped with stimulation electrodes of any shape and/or size (e.g. electrode pads), since the outer surface of the housing sides modular implant device allows for freedom of form. Further, the electrodes can be custom-sized electrodes applied to a ceramic outer surface, instead of an array. Advantageously, large shapes of outer stimulation electrode do not compromise valuable component space inside the modular device. Thus, beside conventional stimulation through electrical leads having tip electrodes at their ends (also referred to lead stimulation), the implant device as described herein also provides the option of performing local stimulation at the exposed surface of the implant itself. The stimulation of tissue using stimulation electrodes (e.g. in the surrounding vicinity of the housing) can also be referred to as local surface stimulation.

Further, at least one modular element may be a circuit module, wherein the circuit module comprises a circuit board enclosed by its frame. Preferably, the circuit board is brazed to the frame, although other means of connecting the board to the frame are possible. In accordance with yet another embodiment, the circuit board is a printed circuit board (PCB), wherein the PCB comprises a ceramic material, and wherein the PCB comprises at least one electric contact and/or at least one feedthrough. The electric contacts are configured for connection with an electric contact tip of one of the leads. The feedthroughs are used to carry a signal through the circuit board, e.g. to anther circuit board. Such a design leads to the dispensability of separate PCBs or feedthroughs, since all components are readily integrated in the circuit modules. The PCB may also contain one or more processors configured for wired or wireless communication with a source located external to the housing. The PCB may include any electric circuit that may be configured to perform a logic operation on at least one input variable. Therefore, the PCB may include one or more integrated circuits, microchips, microcontrollers, and microprocessors, which may be all or part of a central processing unit (CPU), a digital signal processor (DSP), a field programmable gate array (FPGA), or any other circuit that may be suitable for executing instructions or performing logic operations.

It may also be beneficial when the at least one electric contact or the at least one feedthrough of each circuit module is configured for electrical connection with the electrical contact tip of at least one electrical lead disposed on an outer surface of the housing of the modular device stacked just over or just beneath said circuit module. This way, different modular elements can be electrically connected to each other in a simple manner, allowing for a highly modular and flexible way of stacking the modular elements. Thus, any combination of lead stimulation and local surface stimulation is easily achievable with the modular implant device as described herein. Furthermore, either just one housing side (top or bottom) of the housing stack may be functionally used for stimulation (single-sided) or both housing sides may be functionally used for stimulation (double-sided). In a double-sided use, either the same type of stimulation (i.e. lead stimulation or local surface stimulation) may be implemented on both housing sides (e.g. both housing sides are configured for lead stimulation), or different types of stimulation may be implemented on each housing side (e.g. lead stimulation on one housing side and local surface stimulation the other). Lastly, both types of stimulation can be parallelly implemented on the same housing side.

According to another aspect of this disclosure, a system for electrical nerve stimulation is provided, the system comprising a network of at least two modular devices, each device configured for implantation inside a body of a subject, wherein each modular device is electrically connected to at least one other modular device through electrical leads. Such a system may be applicable in any appropriate FES context. For example, the system as described herein may be used for stimulation therapies in cases of head or neck pain, spinal cord injuries, stroke and upper limb recovery, drop foot and hypoglossal stimulation.

### BRIEF DESCRIPTION OF THE DRAWINGS

The accompanying drawings, which are incorporated in and constitute a part of this specification, illustrate several examples of the disclosed subject matter. The drawings depict the following:

FIG. 1 depicts an exploded-view drawing of a modular implant device according to a preferred embodiment;

FIG. 2a depicts a schematic drawing of a single-sided modular implant device according to another embodiment;

FIG. 2b depicts a cutaway drawing of the single-sided modular implant device according to the embodiment of FIG. 2a;

FIG. 3a depicts a schematic drawing of a double-sided modular implant device according to another embodiment;

FIG. 3b depicts a cutaway drawing of the double-sided modular implant device according to the embodiment of FIG. 3a;

FIG. 4a depicts a schematic drawing of a double-sided modular implant device according to another embodiment;

FIG. 4b depicts a cutaway drawing of the double-sided modular implant device according to the embodiment of FIG. 4a;

FIG. 5a depicts a schematic drawing of a modular implant system according to a preferred embodiment;

FIG. 5b depicts a schematic drawing of a modular implant system according to another embodiment.

### DETAILED DESCRIPTION OF THE EXEMPLARY EMBODIMENTS

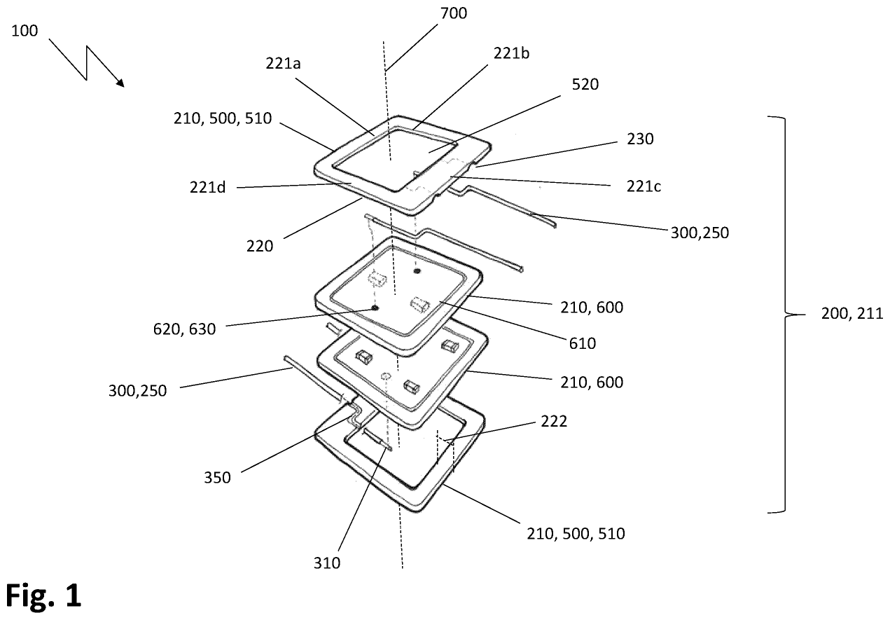

FIG. 1 depicts an exploded-view drawing of a modular implant device 100 according to a preferred embodiment. The modular implant device 100, which is configured for implantation inside a body of a subject, comprises: a housing 200, consisting of two modules hermetically sealed together, and at least one electrical lead 300 and/or at least one stimulation electrode 400 (not shown in FIG. 1). The at least one electrical lead 300 is partly disposed in the housing 200, which comprises modular elements 210 arranged in a stack 211. Such a modular design is highly advantageous, since it may be applied for different product variants of implantable stimulation devices. Furthermore, a stack 211 design has the advantage of simplifying otherwise intricate manufacturing processes. According to FIG. 1, the stacked housing 200 comprises four modular elements 210 attached to each other along a vertical axis 700. The order of modular elements 210 within the stack 211 depicted is not mandatory. Moreover, it is possible that the modular elements 210 be stacked 211 in any other (interchangeable) order, allowing for an even higher flexibility in application. The individual modular elements 210 may be attached to each other via gluing, brazing, welding, or by any other hermetic fixation system.

According to the preferred embodiment, each modular element 210 comprises a frame 220 having four sides 221a,b,c,d, each side 221a,b,c,d having a width 222. In the embodiment shown in FIG. 1, the frame 220 is essentially square. However, other shapes are equally conceivable (e.g. an oblong shape, a circular shape etc.). The frame 220 may at least partly made from a titanium alloy. Providing frames 220 for the modular elements 210 drastically increases stability of the housing 200 and thus of the modular device 100 as a whole. In addition, the frames 220 facility manufacturing of the device 100, since they can serve as a working surface for attaching the modular elements 210 to a stack 211, without having to handle any intricate components. Furthermore, titanium is biocompatible and can therefore be used for implantation in a subject's body without the risk of being rejected or causing inflammations or allergies. Additionally, titanium alloys are highly durable, long lasting, light weight, without compromising strength.

According to the embodiment of FIG. 1, the housing comprises two strain relief modules 500 and two circuit modules 600, wherein the two circuit modules 600 are positioned (“sandwiched”) between the two strain relief modules 500. Each strain relief module 500 comprises a cavity 520 enclosed by the frame 220, wherein the frame 220 is configured as a strain relief ring 510. Such a strain relief module 500 has the advantage of increasing the durability of the modular device 100, since the strain relief module 500 minimizes the risk of lead 300 damages. To further increase durability of the modular implant device 100, the cavity 520 enclosed in the frame 510 of the strain relief module 500 can be backfilled with an insulating material, the insulating material preferentially being silicone.

The circuit modules 600 comprise a circuit board 610 enclosed by the frame 220. The circuit board 610 is preferably brazed to the frame 220. In accordance with a preferred embodiment, the circuit board 610 is a printed circuit board (PCB), wherein the PCB 610 further comprises a ceramic material. Furthermore, the circuit board 610 comprises at least one feedthrough 620. Such a circuit may also be referred to as a hybrid ceramic and requires no separate PCBs or feedthroughs, since all components are readily integrated in the circuit modules. The PCB may include any electric circuit that may be configured to perform a logic operation on at least one input variable. Therefore, the PCB may include one or more integrated circuits, microchips, microcontrollers, and microprocessors, which may be all or part of a central processing unit (CPU), a digital signal processor (DSP), a field programmable gate array (FPGA), or any other circuit that may be suitable for executing instructions or performing logic operations.

The electrical leads 300 as shown in FIG. 1 comprise an electrical contact tip 310 on one of their respective ends. The end comprising the electric contact tip 310 is disposed inside the housing 200, more specifically inside the cavity 520 of a strain relief module 500. Each electric lead 300 is configured to electrically communicate signals from a circuit or power supply 800. As depicted in FIG. 1, the electrical leads 300 pass through lead exits 230 disposed in at least one side 221a,b,c,d of the strain relief ring 510 of the strain relief module 500. To allow for a decent strain relief functionality, the width 222 of a side 221a,b,c,d of the strain relief ring 510 having a lead exit 230 is bigger than the width of a side of the strain relief ring 510 having no lead exit 230. Further, the wider side 221a,b,c,d of the strain relief ring 510 provides more space for the electrical lead 300 to pass through, leading to a higher strain relief factor. In addition, the electrical lead 300 can form a double bend 350 when passing through the lead exit 230 of the strain relief ring 510. Such a double bend 350 prevents the lead 300 from losing contact to a respective circuit board 610 or from being pulled out of the housing 200. Furthermore, the above concept allows for maximum freedom of electrical contacts 630 and lead exit 230 positions and orientation. The strain relief ring 510 can easily adapt for any lead exit 230 location or orientation, giving the modular device 100 a high flexibility with regards to applications in FES.

It may also be beneficial when the at least one electric contact 630 or the at least one feedthrough 620 of each circuit module 600 is configured for electrical connection with the electrical contact tip 310 of at least one electric lead 300 disposed in the cavity 520 of the strain relief module 500 stacked just over or just beneath said circuit module 600. This way, different modular elements 210 may be electrically connected with each other in a simple manner, allowing for a highly modular and flexible way of stacking the modular elements 210. Thus, any combination of lead stimulation and/or local surface stimulation is easily achieved with the modular implant device 100.

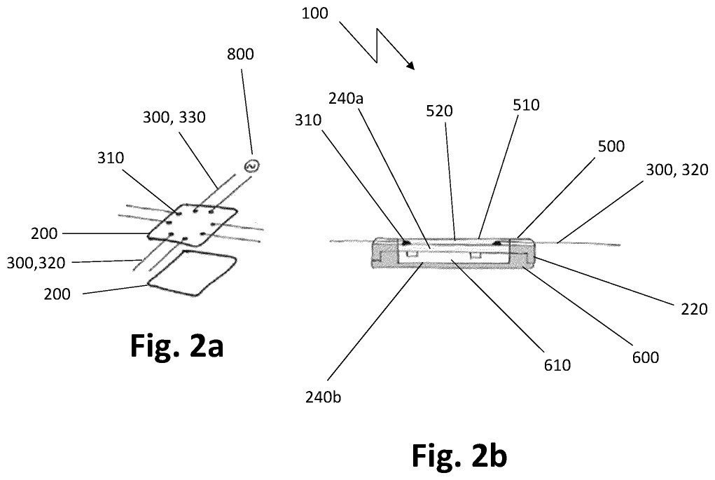

FIG. 2a depicts a schematic drawing of a single-sided modular implant device 100 according to a another embodiment. FIG. 2b depicts a cutaway drawing of the same embodiment. With a modular device 100 as described herein, it is possible to provide a device 100 having one stimulating housing side 240a, i.e. stimulation signals may be generated on just one of the two housing sides 240a or 240b (single-sided). Furthermore, stimulation can be achieved through use of the at least one electrical lead 300, which is then configured as a stimulation lead 320. Each stimulation lead 320 comprises a tip electrode at one of its ends that is not disposed inside the housing 200 of the modular device 100, but that is instead positioned in a desired location with respect to the tissue to be stimulated. The stimulation of tissue using stimulation leads (e.g. in a location further away from the housing) can also be referred to as lead stimulation. Stimulation via a stimulation lead 320 is also referred to as lead stimulation. Alternatively or additionally, stimulation may be achieved thought use of the at least one stimulation electrode 400. Stimulation via stimulation electrode 400 is also referred to as local surface stimulation.

The embodiment as depicted to FIG. 2a and FIG. 2b is single sided, meaning that only one housing side 240a generates a stimulation signal. The embodiment shown in the figures uses lead stimulation, i.e. it comprises several stimulation leads 320 that stimulate at least one nerve or muscle of the subject in a location away from the housing 200. The stack 211 order of modular elements 200 implemented in the embodiment shown in FIG. 2b is as follows: a circuit module 600 as the bottom side and a strain relief module 500 as the top side.

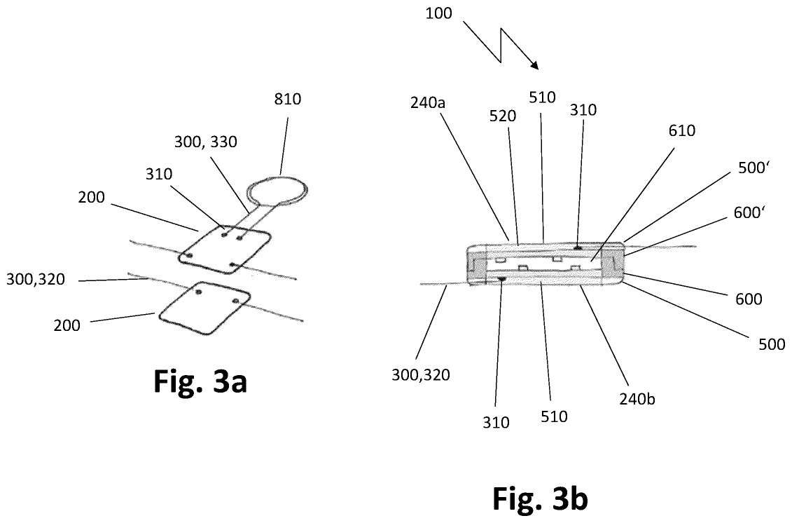

The embodiment as shown in FIG. 3a and FIG. 3b is configured to be a double-sided modular implant device 100, meaning both of its housing sides 240a and 240b are able to generate stimulation signals. As depicted, the type of stimulation is lead stimulation, with the strain relief module 500 of each housing side 240a,b comprising two stimulation leads 320. Furthermore, the top housing side 240a comprises two additional electrical leads that are power leads 340 configured for delivering power from an implanted coil 810 receiving power and potentially data (in a transcutaneous system) from an power supply 800. The stack 211 order of modular elements 200 implemented in the embodiment shown in FIG. 3b is as follows: a first strain relief module 500, a first circuit module 600 and a second circuit module 600′ arranged in the middle of the stack 211 and a second strain relief module 500′.

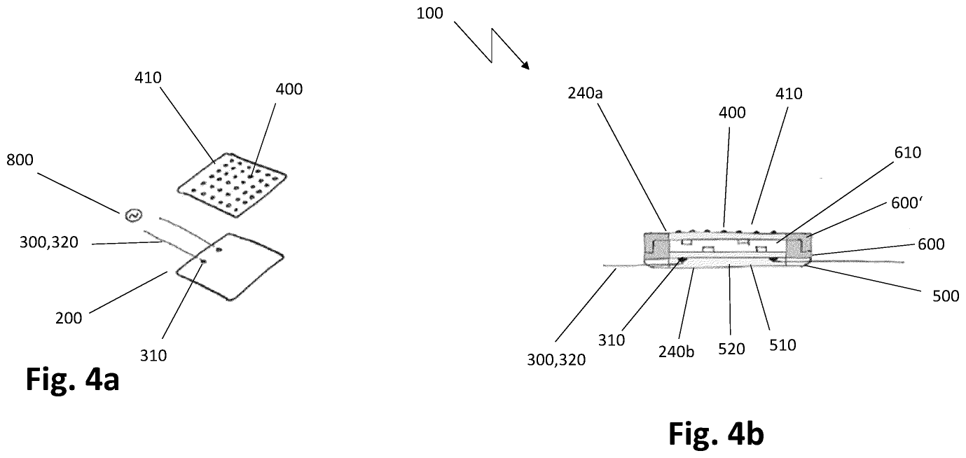

The embodiment as shown in FIGS. 4a and 4b is also configured to be a double-sided modular device, meaning both of its housing sides 240a,b are enabled to generate stimulation signals. However, in contrast to the embodiment depicted in FIG. 3a and FIG. 3b, the top housing side 240a comprises stimulation electrodes 400 attached to its outer surface. In this embodiment, the modular device 100 comprises a plurality of stimulation electrodes 400, wherein the plurality of stimulation electrodes 400 is arranged in an array 410. If an array 410 is packed sufficiently densely with stimulation electrodes 400, it is, for example, suitable, for example, for being used in scanning applications. The stack 211 order of modular elements 200 used in the embodiment shown in FIG. 4b is as follows: a strain relief module 500 as the bottom housing side, a first circuit module 600 in the middle and a second circuit module 600′ as the top housing side 240a, with the electrode array 410 being disposed on the outer surface of the second (top housing side 240a) circuit module 600′. The bottom housing side 240b of the modular device 100 of FIG. 4b is configured for lead stimulation.

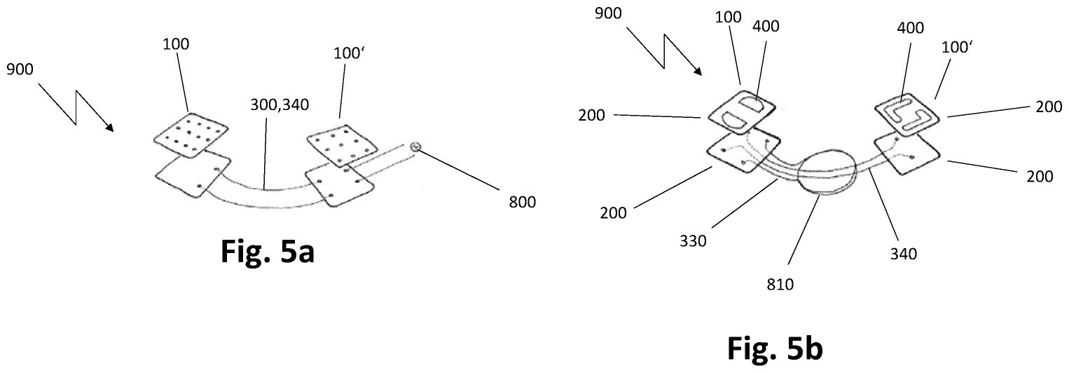

FIG. 5a depicts a schematic drawing of a modular implant system 900 according to a first embodiment. Furthermore FIG. 5b depicts a schematic drawing of a modular implant system 900 according to a second embodiment. A system 900 for electrical nerve stimulation as shown in FIG. 5a and FIG. 5b comprises a network of at least two modular implant devices 100, 100′ configured for implantation inside a body of a subject, wherein each modular device is electrically connected to at least one other modular device through electrical leads. Further, according to the embodiment shown in FIG. 5b, the electrodes 400 can be of variable shape and or formations, e.g. circular, half-circular, planar, in an array etc.

The invention is not limited to one of the embodiments described herein but may be modified in numerous other ways.

All features disclosed by the claims, the specification and the figures, as well as all advantages, including constructive particulars, spatial arrangements and methodological steps, can be essential to the invention either on their own or by various combinations with each other.

List of reference numerals

| | |
|---|---|
| Modular implant device | 100 |
| Housing | 200 |
| Modular element | 210 |
| Stack | 211 |
| Frame | 220 |
| Side | 221a, b, c, d |
| Width | 222 |
| Lead exit | 230 |
| Top housing side | 240a |
| Bottom housing side | 240b |
| Electrical lead | 300 |
| Electrical contact tip | 310 |
| Stimulation lead | 320 |
| Power lead | 330 |
| Connection lead | 340 |
| Double bend | 350 |
| Stimulation electrode | 400 |
| Array | 410 |
| Strain relief module | 500 |
| Strain relief ring | 510 |
| Cavity | 520 |
| Circuit module | 600 |
| Circuit board | 610 |
| Feedthrough | 620 |
| Electric contact | 630 |
| Vertical axis | 700 |
| Power supply | 800 |
| Antenna coil | 810 |
| System | 900 |

1. A modular implant device configured for stimulation of at least one nerve or muscle in a body of a subject, the device comprising:
a housing; and
at least one electrical lead and/or at least one stimulation electrode;
wherein the at least one electrical lead is partly attached to the housing, and wherein the housing comprises modular elements, the modular elements being arranged in a stack.

2. The modular implant device according to claim 1, wherein the stack comprises at least two modular elements which are hermetically sealed and attached to each other.

3. The modular implant device according to claim 1, wherein each modular element comprises a frame.

4. The modular implant device according to claim 3, wherein the frame is four sided, and each side has a width.

5. The modular implant device according to claim 3, wherein the frame is at least partly made from a material comprising titanium.

6. The modular implant device according to claim 3, wherein at least one modular element is a strain relief module, the strain relief module comprises a cavity enclosed by the frame and wherein the frame is configured as a strain relief ring.

7. The modular implant device according to claim 6, wherein the cavity is backfilled with an insulating material.

8. The modular implant device according to claim 7, wherein the insulating material comprises silicone.

9. The modular implant device according to claim 1, wherein the at least one electrical lead comprises an electrical contact tip on at least one of its ends.

10. The modular implant device according to claim 9, wherein the electrical contact tip is disposed on the housing, wherein the electrical contact tip is in particular disposed in the cavity of one of the strain relief module.

11. The modular implant device according to claim 4, wherein the electrical lead passes through a lead exit disposed in one of the sides of the strain relief ring of the strain relief module.

12. The modular implant device according to claim 11, wherein the width of a side of the strain relief ring having a lead exit is bigger than the width of a side of the frame having no lead exit.

13. The modular implant device according to claim 11, wherein the electrical lead forms a double bend when passing through the lead exit of the strain relief ring.

14. The modular implant device according to claim 1, wherein at least one of the electrical leads is a stimulation lead configured for electrical stimulation of a nerve of the subject, or a power lead configured for delivering power from a power supply and/or for delivering power and data from an antenna coil, or a connection lead configured for electrically connecting the modular device to another electrical device.

15. The modular implant device according to claim 1, wherein the at least one stimulation electrode is disposed on an outer surface of a housing side of the modular device.

16. The modular implant device according to claim 1, wherein the modular device comprises a plurality of stimulation electrodes.

17. The modular implant device according to claim 16, wherein the plurality of stimulation electrodes is arranged in an array.

18. The modular implant device according to claim 3, wherein at least one modular element is a circuit module, wherein the circuit module comprises a circuit board enclosed by the frame.

19. The modular implant device according to claim 18, wherein the circuit board is brazed to the frame.

20. The modular implant device according to claim 18, wherein the circuit board is a printed circuit board, wherein the circuit board comprises a ceramic material.

21. The modular implant device according to claim 18, wherein the circuit board comprises at least one electrical contact and/or feedthrough.

22. The modular implant device according to claim 21, wherein the at least one feedthrough of each circuit module is configured for electrical connection with the electrical contact tip of at least one electrical lead disposed in the cavity of the strain relief module stacked just over or just beneath said circuit module.

23. A system for electrical nerve stimulation, the system comprising a network of at least two modular devices configured for implantation inside a body of a subject, wherein each modular device is electrically connected to at least one other modular device through electrical leads, and wherein each of the at least two module devices comprises a housing; and at least one electrical lead, and/or at least one stimulation electrode; and wherein the at least one electrical lead is partly attached to the housing, and wherein the housing comprises modular elements, the modular elements being arranged in a stack.
